(19) United States
(12) Patent Application Publication
(10) Pub. No.: US 2017/0159076 A1
(43) Pub. Date: Jun. 8, 2017

(54) METHOD FOR FRACTIONATING A LIGNOCELLULOSIC BIOMASS

(71) Applicant: Arbiom Inc., Durham, NC (US)

(72) Inventors: Naga Vinod Kumar Bathula, Norton, VA (US); Lisette Tenlep, Big Stone Gap, VA (US); William F. McDonald, Utica, OH (US); Fan Hu, Atlanta, GA (US); Aziz Ahmed, Chesterfield, MO (US)

(73) Assignee: Arbiom Inc., Durham, NC (US)

(21) Appl. No.: 15/368,648

(22) Filed: Dec. 4, 2016

Related U.S. Application Data
(60) Provisional application No. 62/263,589, filed on Dec. 4, 2015.

(51) Int. Cl. C12P 7/10

(52) U.S. Cl. CPC C12P 7/10; C12P 2201/00

(57) ABSTRACT

The present invention relates to a method for fractionating a lignocellulosic biomass. More specifically, the present invention lies in the field of cellulose solvent- and organic solvent-based lignocellulose fractionation (COSLIF) of lignocellulosic biomasses. In this type of process, a cellulose solvent, such as phosphoric acid or an ionic liquid is used to disrupt the structure of cellulose in a lignocellulosic biomass. Subsequent washing and treatment steps are used to fractionate the biomass.

The present invention relates to a method for fractionating a lignocellulosic biomass. More specifically, the present invention lies in the field of cellulose solvent- and organic solvent-based lignocellulose fractionation (COSLIF) of lignocellulosic biomasses. In this type of process, a cellulose solvent, such as phosphoric acid or an ionic liquid is used to disrupt the structure of cellulose in a lignocellulosic biomass. Subsequent washing and treatment steps are used to fractionate the biomass.

### BACKGROUND OF THE INVENTION

The consumption of non-renewable resources in the world is continuing to increase in accelerating rate with the economic advancement of once developing and under developing countries to catch up with developed countries. It is important to find or develop a renewable energy sources to continue the economic advancement for the future generation. Billions of tons of biomass that include trees, agricultural residues, and many woody and non-woody plants are available every year to meet the future demand of renewable energy sources. All biomass consists of (at least) three basic constituents, cellulose, hemicellulose and lignin at various ratios depending on the types of biomass. Research is going on around the world during the last few decades to develop an economically viable process to convert biomass into fuel and various bio-based products which are now produced from non-renewable resources such as coal, petroleum, etc. Both thermal and biological process can be used to convert biomass into fuel and bio-products. In general, the conversion of biomass into biofuel and bio-based products using thermal and biological process involve biomass processing into smaller particles, pretreatment at alkaline or acidic conditions using various chemicals at various temperatures for various residence times. The purpose of the thermo-chemical pretreatment of biomass is to disrupt the biomass structure up to the individual fiber level to reduce the cellulose crystallinity to a level to increase the accessibility of biofuel producing microbe into the fiber matrix to complete the job.

Instead of refining fossil fuels to make hydrocarbon derivatives, biorefinery processes rely on the degradation of biomass, such as wood, agricultural and paper wastes into fuel, fiber, energy and sugars. In turn, sugars are raw materials that can be used for making many products, such as bioplastics, ethanol, acetic acid and other chemicals. With a global decline in fossil fuel stores, biorefinery processes are becoming increasingly important, and biomass-derived products increasingly gain economic and environmental advantages over products which are produced from fossil fuels. In many instances, the basis for a biorefinery process is a lignocellulosic biomass derived from lignocellulosic containing plants the biomass of which finds its way into products, such as wood, agricultural residues and paper as well as paper wastes. Lignocellulosic biomass is a polymeric material containing cellulose, lignin and hemicellulose. Cellulose is polymer composed of chains of six-carbon sugars, which chains are bundled into strong fibers which have areas of more or less crystalline structure. Lignin is a complex, three dimensional polymer composed of linked six-carbon-phenolic rings with various carbon chains and other chemical functionalities. Lignin is non-crystalline, and its structure has been described as analogous to a gel or foam. Lignin serves to bind the cellulose fibers. Hemicellulose is a complex polymer composed of various five- and six-carbon sugars in a highly branched structure. Hemicellulose bonds weakly to both cellulose and lignin and fills the intervening spaces in the lignocellulosic biomass.

When a lignocellulosic biomass is separated into its three fractions cellulose, hemicellulose and lignin, each of such fraction has a wide range of uses and by-products, including sugars, ethanol, and other chemical products. The cellulose fraction has potential use as fiber or as a feedstock for the production of fuel-grade ethanol. The resulting lignin fraction has potential as a source of specialty chemicals, but in the near term, as a fuel for co firing with coal or natural gas to produce electricity at the biorefining side. The hemicellulose fraction has potential for being hydrolysed into five-carbon sugars, which are building blocks for numerous products and industrial chemicals or producing energy by anaerobic digestion which converts the sugars and non-sugars to methane.

In the past, lignocellulosic biomass has been degraded by various means, including enzymatic means, chemical means, alone or in conjunction with physical means. Enzymatic hydrolysis has been used in the past but its use has been limited by the costs of the appropriate cellulose degrading enzymes. This is because in the presence of lignin enzymes are blocked from reaching certain sites and may become irreversibly bound to the lignin. As a result of this non-specific and irreversible binding, the enzymes are consumed in the course of the process rather than functioning as proper catalysts. Chemical means that have been used to break down lignocellulose include acids, alkali, oxidation, other chemical reactions, alone or in conjunction with heat. One of the challenges for fractionation of lignocellulosic biomass using phosphoric acid is to break up the larger polymeric structures while preserving the building blocks for various downstream uses.

Another approach to dissolving the biomass process is the use of ionic liquid solvents to dissolve the biomass or certain components from the biomass. The major drawback from using the ionic liquids is their expense

Among the many pretreatment processes under investigation, the use of concentrated phosphoric acid in pretreatment has drawn attention in recent years. The process requires low temperature at atmospheric pressure contraire to some high temperature and high pressure pretreatment.

WO 2007/111605 describes a cellulose-solvent-based lignocellulose fractionation with modest reaction conditions and alleged reagent cycling. The process disclosed in WO 2007/111605 aims at the production of highly amorphous cellulose which can be converted into glucose, furthermore at the production of hemicellulose, lignin and acetic acid. The process employs chemical means to dissolve the lignocellulosic biomass into cellulose, hemicellulose, lignin and acetic acid. A similar process is described in WO 2009/114843. In both documents, the lignocellulosic biomass is exposed to an acidic solvent to perform a solvation and/or dissolution process, subsequent to which the resultant product is transferred into a separate reactor to be treated with further chemicals. The processes described in these documents, whilst showing some advantages with respect to earlier processes, do not allow for precise process control, especially not in the dissolving step, and in many instances result in a solvation or dissolution that has either gone too far or has not progressed sufficiently far enough. If the solvation or dissolution process has progressed too far, the problem is that there are too many dissolved components which get subsequently simply washed out from the reaction. If the dissolution had not progressed sufficiently far enough, this results in the reaction mass not dissolving and becoming difficult to transfer to the next reaction or separation vessel where further process steps are to take place. At worst, the reaction mass will be a solid cake that is hard to transfer to the next vessel and will have to be manually removed from the first reactor. Also, the processes described in these prior art documents do not allow a recovery of the various components contained in the lignocellulosic biomass, let alone a recycling of the various chemicals and solvents used.

EP 2304058 A1 described a process of decrystallization of cellulosic biomass with an acid mixture comprising phosphoric acid and sulfuric acid to enhance the enzyme digestibility of treated biomass. However, the document does not disclose any concept of recovery or recycle of phosphoric acid or sulfuric acid.

A., Goshadrou, K., Karimi, et al. has described a process of enhancing non-isothermal saccharification and fermentation (NSSF) for ethanol production by phosphoric acid pretreatment of aspen wood chips. Acetone has been used as amorphous cellulose regenerating solvents. However, the authors have not cited, suggested or described any process of chemical recovery from the process.

Isroi, M. M. Ishola, et al. has treated oil palm empty fruit bunch (OPEFB) using white-rot fungus, phosphoric acid or their combination, and the results were evaluated on the biomass components, and its structural and morphological changes, and the digestibility of the pretreated biomass. The experimental results showed that the fungal-, phosphoric acid-, and fungal followed by phosphoric acid pretreatments have improved the digestibility of OPEFB's cellulose by 4, 6.3 and 7.4 folds respectively. However, an acid recovery method was not included in this study.

J. Zhang, J. Zhang, et al. has described a process of dissolving microcrystalline cellulose (MCC) with phosphoric acid to obtain high-quality fermentable saccharides. The authors also studied the molecular changes in MCC and kinetics of the reaction during the phosphoric acid pretreatment. Phosphoric acid recovery and solvent recovery process were not cited in the paper.

CN 102690899 A describes a method for hydrolyzing lignocellulose raw material by concentrated phosphoric acid and recovering phosphoric acid. The document discloses a model method comprising of the following steps: hydrolyzing a lignocellulose raw material in concentrated phosphoric acid at the temperature of 50-200° C. to obtain water soluble sugar; separating a solution containing the soluble sugar from solid residue by filtration or centrifugation; adding an organic solvent, which accounts for 1-10 times the volume of the concentrated phosphoric acid and is slightly soluble or water soluble at room temperature, into the solution containing the soluble sugar, mixing, separating by filtration or centrifugation to obtain soluble sugar precipitate and an organic solvent containing phosphoric acid; and separating the organic solvent containing phosphoric acid by distillation or back extraction with water to obtain a phosphoric acid solution and organic solvent. According to the document, however, the whole process seems to be a simulated one without any real experimental data. The prior art processes described in this field do not allow for an efficient recovery and recycling of the various solvents involved, nor was it possible to obtain a purified lignin fraction.

Accordingly, there is a need in the art for an improved process of a method of fractionation of a lignocellulosic biomass which allows for an efficient recovery and recycling of the chemicals used thus increasing the overall efficiency of the process.

### SUMMARY OF THE INVENTION

Accordingly, in a first aspect, the present invention relates to a method for fractionating a lignocellulosic biomass, comprising the steps:
a) providing a lignocellulosic biomass and pretreating said lignocellulosic biomass with phosphoric acid;
b) exposing said pretreated lignocellulosic biomass to phosphoric acid for a defined period of time at a defined temperature range to dissolve cellulosic and/or hemicellulosic components of said biomass;
c) quenching, within the same reaction vessel as for step b), the dissolution of said cellulosic and/or hemicellulosic components, by adding a first solvent to said lignocellulosic biomass, wherein said first solvent is water, an alcohol, an ether, a ketone or a mixture of any of the foregoing, thereby precipitating said cellulosic and hemicellulosic components as solids,
d) separating said precipitated solids, preferably amorphous solids, of step c) from a liquid fraction comprising said phosphoric acid and said first solvent of step c), washing said separated precipitated solids, preferably amorphous solids, by a second solvent which is water, an alcohol, an ether, a ketone or a mixture of any of the foregoing, and combining said liquid fraction with said second solvent that has been used for washing said separated precipitated solids, preferably amorphous solids, to form a combined liquid fraction, said combined liquid fraction comprising said phosphoric acid, said first and second solvent and lignin components dissolved in any of said phosphoric acid, said first or said second solvent;
e) evaporating said first and said second solvent from said combined liquid fraction, thereby producing a concentrated combined liquid fraction, and, optionally, recycling said first and second solvents, after condensation thereof, to be re-used in step c) and/or d);
f) adding water to said concentrated combined liquid fraction, to precipitate said lignin components dissolved in said concentrated combined liquid fraction, such as lignin, thus producing a precipitate of said lignin components, and a dilute combined liquid fraction, said dilute combined liquid fraction comprising phosphoric acid and water;
g) separating said precipitate of said lignin components from said dilute combined liquid fraction, thus producing a solid crude lignin fraction and, separately therefrom, said dilute combined liquid fraction;
h) concentrating said dilute combined liquid fraction by evaporating said water therefrom, thus producing concentrated phosphoric acid; and
i) recycling said concentrated phosphoric acid of step h) to steps a) and/or b).

In one embodiment, said evaporation in step e) occurs at a pressure <1 bar and at a temperature below the boiling point(s) of said first and second solvent, preferably <78° C., preferably in the range of from 20° C. to 78° C.

In one embodiment, said water that is added in step f) has a temperature that is equal or higher than the temperature of said concentrated combined liquid fraction and that is in the range of from 20° C. to 90° C.

In one embodiment, the mass ratio of water to concentrated combined liquid fraction in step f) is in the range of from 1:1 to 10:1, preferably 1:1 to 5:1, preferably 1:1 to 4:1, more preferably 2:1 to 3:1.

In one embodiment, said concentrated phosphoric acid obtained in step h) is mixed with fresh concentrated phosphoric acid and is thereafter recycled in step i) as such mixture to be used in step a) and/or b). Preferably, such fresh acid is used to cover losses in the acid recovery process.

In one embodiment, said first and said second solvent are independently, at each occurrence, selected from water, methanol, ethanol, isopropanol, 1-propanol, 1-butanol, 2-butanol, t-butanol, isobutanol, fatty alcohol, acetone, butanone, pentanone, dimethylether, methylether, diethylether, tetrahydrofurane, and mixtures of any of the foregoing.

In one embodiment, in step f), additionally, a flocculant is added.

In one embodiment, said flocculant is mainly an anionic flocculant. Various types of flocculants of anionic, cationic and non-ionic nature can be used.

In a preferred embodiment, the flocculant is an anionic flocculant or a polyacrylamide-based flocculant, in particular a polyacrylamide-based anionic flocculant. Examples are Nalclear® 7763 or Optimer® 9825.

In one embodiment, said flocculant is added in an amount of 0.05 wt. %-2 wt. %, preferably 0.05 wt %-1 wt %, more preferably 0.1 wt % to 0.5 wt %, with reference to the weight of said concentrated combined liquid fraction. A preferred and useful concentration is 0.2 wt. % flocculant.

In one embodiment, said first and second solvent are the same. In one embodiment, the first and second solvents are ethanol (EtOH) or water.

In one embodiment, in the pretreatment step a), the weight ratio of said phosphoric acid to said lignocellulosic biomass is in the range of from 1:1 to 13:1, preferably 2:1 to 8:1, more preferably 4:1 to 6:1.

In one embodiment, said pretreating occurs for a time period of 1 min to 30 h, preferably 1 h to 5 h, and/or at a temperature in the range of from 0° C. to 100° C., preferably 25° C. to 60° C.

In one embodiment, step b) is performed for a period of 30 min to 5 h, preferably 1 h to 5 h, and at a temperature higher than the temperature of the pretreating step a), preferably in the range of from 40° C. to 90° C., preferably 50° C. to 80° C., more preferably 60° C. to 70° C.

In one embodiment, the weight ratio of said first solvent to said biomass in step c) is in the range of from 12:1 to 5:1, preferably 10:1 to 6:1.

In one embodiment, the weight ratio of said second solvent to said biomass in step d) is 30:1 to 2:1, preferably 25:1 to 6:1.

In one embodiment, the method according to the present invention further comprises the additional first step: washing the precipitated solids washed in step d), by water at a weight ratio of water:biomass of 200:1 to 50:1, preferably 150:1 to 100:1

In one embodiment, the method according to the present invention further comprises the additional second step: subjecting the water washed solids of the additional first step to hydrolysis, preferably enzymatic hydrolysis, more preferably by cellulase(s) and/or hemicellulase(s), and subjecting the product of such hydrolysis subsequently to fermentation.

In one embodiment, the method according to the present invention further comprises the additional step: subjecting the solid crude lignin fraction of step g) to an acidic hydrolysis using water, preferably at a temperature in the range of from 50° C. to 100° C., said acidic hydrolysis being optionally preceded by one or several washing steps using water said hydrolysis resulting in a solid fraction and a liquid fraction, separating said solid fraction and said liquid fraction from each other, washing the solid fraction and drying it, wherein said liquid fraction is an aqueous solution of sugars, including sugar monomers and oligomers, and said solid fraction is clean lignin. The residual acid in the solids makes the material acidic so, typically, the addition of water for the final hydrolysis results in an acidic hydrolysis.

### DESCRIPTION OF THE PREFERRED EMBODIMENTS OF THE INVENTION

Embodiments of the method according to the present invention provide for a pretreatment of the lignocellulosic biomass with phosphoric acid, subsequent to which the thus pretreated lignocellulosic biomass is exposed to phosphoric acid to dissolve cellulosic and/or hemocellulosic components. Thereafter, such dissolution is quenched in the same reaction vessel as for the previous step (i. e. where the dissolution took place), by adding an appropriate first solvent. The fact that the pretreatment with phosphoric acid, the exposure to phosphoric acid and the quenching take place in the same vessel, already is a substantial improvement over the prior art, where these steps were typically performed in separate vessels, thus adding to the complexity and logistics of the process. An appropriate first solvent may be water, an alcohol, an ether, a ketone, or a mixture of any of these. A typical example is ethanol. As a result thereof, the cellulosic and hemicellulosic components will precipitate as solids, preferably amorphous solids. Subsequently, these precipitated solids, preferably amorphous solids, can be separated from the liquid fraction present, and can be washed using a second solvent which also is water, an alcohol, an ether, a ketone or a mixture of any of the foregoing. In some embodiments, the first and the second solvents are the same, i. e. both are water, an alcohol or an ether or a ketone. In a preferred embodiment, the first and the second solvent are ethanol. The second solvent which is used for washing is subsequently combined with the liquid fraction resulting from the quenching step, and this resultant “combined liquid fraction” comprises phosphoric acid, the first solvent and the second solvent and, additionally, lignin components dissolved in any of the aforementioned solvents. This “combined liquid fraction” is dark in colour and is also sometimes referred to herein as “black liquor”. In accordance with embodiments of the present invention, the combined liquid fraction (i. e. the “black liquor”) is subjected to an evaporation step by which the first and the second solvent are evaporated from the combined liquid fraction. Such evaporation is preferably performed at reduced pressure, i. e. below 1 bar and at a temperature below the boiling point(s) of the first and the second solvent. If the first and second solvent are identical, then such evaporation is preferably performed below the boiling point of such solvent. The evaporation step will result in a “concentrated combined liquid fraction”, i. e. a “concentrated black liquor”. The “concentrated combined liquid fraction” is thus a liquid fraction comprising phosphoric acid, lignin components dissolved therein and, possibly, other biomass degradation products generated during pretreatment and/or during evaporation of first solvent and second solvent. In a subsequent step, water is added to such concentrated black liquor to precipitate at least part of the lignin components, in particular the crude lignin components, which have been dissolved in said concentrated combined liquid fraction. By the addition of water to such concentrated combined liquid fraction the dissolved lignin components precipitate. What results from this therefore is a precipitate of the lignin components and a dilute combined liquid fraction. Such “dilute combined liquid fraction” comprises phosphoric acid, and water and, possibly some biomass degradation products. The two components, i. e. the dilute combined liquid fraction and the precipitate of the lignin components are subsequently separated. The dilute combined liquid fraction is then concentrated by evaporation of the water. This will result in concentrated phosphoric acid which can be subsequently recycled to be used again in step a) and/or step b). In a preferred embodiment, this concentrated phosphoric acid resulting from such concentration step, is mixed with pristine or fresh phosphoric acid, i. e. phosphoric acid which had previously not been used in the aforementioned process (nor in any other process, and comes “straight from the bottle”), and it is such acid mixture of recycled and pristine (or fresh) phosphoric acid that is then subsequently used in steps a) and b). In a preferred embodiment, the ratio at which the concentrated phosphoric acid of step h) is mixed with fresh concentrated phosphoric acid is approximately in the range of 4:1-9:1.

The method according to the present invention allows for an efficient recycling of both the phosphoric acid and of the first solvent and second solvent. Moreover, the yield of the overall process as well as the purity of the lignin recovered is exceptionally high. In some embodiments, a flocculant is used in order to increase the precipitation of the lignin components dissolved in the combined liquid fraction (i. e. in the black liquor”). The use of such flocculant not only increases the size of the lignin particles to facilitate the filtration efficiency, but also increases the lignin yield as well as the yield of the acid, when compared with a method using no flocculants. In addition to this, the use of flocculant increases the purity and decreases the viscosity of the recycled acid. The viscosity of recycled acid is 49 cSt (=49 mPas) compared to 47 cSt (=47 mPas) for pristine phosphoric acid.

In a further embodiment, the present invention also provides for a recycling of the first and second solvent obtained in the evaporation step e). Such first and second solvent is condensed and is subsequently recycled to be reused in steps c) and/or d). The efficiency of recycling can be further enhanced if the precipitated cellulosic solids of step d) having been washed using the second solvent, are further washed by water, and the resultant liquid fraction is collected. Such liquid fraction contains water, some first and second solvent and phosphoric acid. This is also sometimes herein referred to as “water washate”, and this can be further treated by evaporating the first and second solvent in a first step and by evaporating water in a second step. This will result in a clean fraction of first and/or second solvent, a clean water fraction and a clean concentrated phosphoric acid fraction. The resultant solids of such water washing step are cellulosic solids, and these can then be subjected to enzymatic (or other) hydrolysis, to produce sugars, either in monomeric or oligomeric form or in mixtures thereof, and these sugars can subsequently be subjected to fermentation.

The flocculants that are used in embodiments of the present invention are manifold. They may be anionic, cationic or non-ionic. One example are anionic flocculants. Also preferred are polyacrylamide flocculants, in particular anionic polyacrylamide flocculants. Examples of the products tried are presented in Table 1. The use of a flocculant increases the yield of clean acid and of lignin. The flocculant samples were supplied by Nalco Inc for testing the suitability of the sample for crude lignin precipitation work. Table 1 below shows the applicability of various anionic and cationic flocculants on crude lignin precipitation work. The amount of flocculant doses, amount of water use, temperature of precipitation process have been optimized for both Nalclear® 7763 and Optimer® 9825.

TABLE 1
Identification of suitable flocculants

| Name of | Types of | | Agglomeration of |
|---|---|---|---|
| Flocculant | flocculant | Active constituents | precipitates? |
| Nalclear 7763 | Anionic | Anionic acrylamide | Yes |
| | flocculants | copolymer | |
| Optimer 9825 | Anionic | Ultra high MW, | Yes |
| | Flocculants | moderately anionic | |
| | | liquid flocculants | |

The term “phosphoric acid” is meant to include orthophosphoric acid as well as condensed polymeric forms thereof, such as pyrophosphoric acid and polyphosphoric acid, i.e. HO(PO₂OH)ₓH, where x is the number of PO₂OH-units in the molecule. Also included in the term “phosphoric acid” are cyclic structures forming metaphosphoric acid molecules. This includes examples, such as trimetaphosphoric acid or cyclotriphosphoric acid, i.e. H₃P₃O₉. Also included in such term are the respective phosphoric acid anhydride compounds, such as P₄O₁₀, often abbreviated as P₂O₅. Upon addition of water, such phosphoric acid anhydride will form phosphoric acid.

The term “alcohol” is meant to refer to any hydrocarbon compound having an OH-group and having the general formula R—OH and being a liquid at a temperature in the range of from approximately −140 degrees C. to approximately 250 degrees C. In a preferred embodiment, the term alcohol refers to molecules, such as methanol, ethanol, 1-propanol, 2-propanol, 1-butanol, 2-butanol, t-butanol, isobutanol, but also includes alcohol molecules of the general formula HO—R—R′OH and HO—R—R′OH—R″OH with two or more hydroxy groups, such as ethylene glycol or glycerol. The alcohols listed are not meant to be limiting rather to be descriptive of the potential to use any of a number of different alcohols that are mono-, di-, tri- and multi-functional-. i.e. alcohols that have one, two, three or a plurality of OH-functionalities in their molecular structure.

The term “ketone”, as used herein, is meant to include compounds having a non-terminal carbonyl group in their structure. Examples for a ketone are acetone, butanone, pentanone, methyl ethyl ketone and methyl isobutyl ketone. The ketones listed are not meant to be limiting rather to be descriptive of the potential to use any of a number of different ketones with various ketone structures that can be used to cause the precipitation of the polysaccharides.

The term “ether”, as used herein, is meant to include compounds that contain an ether group, i.e. an oxygen atom connected to two alkyl or aryl groups thus making the general formula R—O—R′. Typical examples of ethers useful according to the present invention are dimethyl ether, methyl ethyl ether and diethyl ether. The ethers listed are not meant to be limiting rather to be descriptive of the potential to use any of a number of different ethers with various ether structures that can be used to cause the precipitation of the polysaccharides.

The term “black liquor” is meant to refer to the liquid mixture or combined liquid mixture of first and second solvent, phosphoric acid, and biomass components after liquid solid separation of step d).

In one embodiment, the first and second solvent contains no water or no more water than in a range of from 0.1% (v/v) to 10% (v/v). In one embodiment, the second solvent is an alcohol, preferably ethanol, more preferably 90%, 91%, 92%, 93%, 94%, 95% or 96% or 97% or 98% or 99% (v/v) ethanol.

The term “x % alcohol”, as used herein, refers to an alcoholic solution which contains x % alcohol and (100-x) % of another solvent, for example water. Typically, a “95% ethanol” is an ethanolic solution containing 95% ethanol and 5% water or 5% denaturant on a volume basis or v/v.

In one embodiment, steps a)-d) are performed without a separate addition of water. By the term “without a separate addition of water” a situation is referred to wherein water is not added to the reaction as a separate solvent. Although, a certain amount of water may be present in the reaction vessel(s) during steps a)-d), due to the possibly inherent water content of lignocellulosic biomass, i.e. such biomass itself containing some water and/or due to the first and/or second solvent possibly containing a small amount of water which is in the range of from 0.1% (v/v) to 15% (v/v), with reference to the amount of phosphoric acid, first or second solvent added, there is no separate addition of water.

In one embodiment, the reaction vessel in which exposure to the acid and the first solvent occurs is a mixing vessel or mixing reactor, a continuously stirred tank reactor (CSTR), a horizontal reactor tube, planetary mixer, extruder, double arm mixer, blender, etc., and is sometimes also referred to as a “mixer”. Again, the list is meant to be a descriptive rather than a limiting list.

Typically, in embodiments according to the present invention, steps which involve a separation into a solid component and a liquid component, such as a solvent, may be performed in a suitable device, e.g. a separation vessel or separation unit, such as a filter, a press, screw press, a basket or filtering centrifuge, a decanting centrifuge, a DSM screen, a wedge wire screen, or similar suitable filtration or screening device. A filter system may be operated under a vacuum, with pressure or using gravity.

The term “to quench” as used herein, is meant to refer to an action of a solvent which slows down, or halts a reaction. Such quenching is typically or in many instances also accompanied by a precipitation of some components.

As used herein, term “lignocellulosic biomass” is meant to refer to a biomass which contains, inter alia, cellulose, hemicellulose, lignin, water extractives, ethanol extractives, acetic acid, phenolic compounds, free sugars (e.g. glucose and sucrose), inorganic minerals, resins, rosins, tannins, etc. The “lignocellulosic biomass” according to the present invention may be derived from different materials, selected from hardwood, softwood, paper, in particular recycled paper, waste paper, wood shavings, sawdust, forest trimmings, pulp, corn stover, corn cobs, corn

fiber, straw, in particular wheat straw, barley straw, rice straw, sugar cane bagasse, switch grass, empty fruit bunches from palm oil trees/residues, forestry thinnings, any sort of native or non-native grass, energy crops, agricultural residues, manure from animal feeding operations, human waste, construction debris, and combinations thereof. There may be economic and supply concerns that cause the use of a certain biomass material rather than others. For instance, in the state of Iowa in the US, corn stover or soybean straw are good candidates for feeding a commercial scale plant, however, forestry thinnings would not be. In contrast, in northern Minnesota, forestry thinnings might provide adequate supply of biomass, but there would be no significant corn or soy residues available.

As used herein, the term “water” may refer to water which, optionally, has dissolved material in it, such as salts.

Typical “reaction vessels” according to the present invention include but are not limited to a reactor, a tank, preferably having at least one stirring device, more preferably having at least two stirring devices, a continuously stirred tank, a tubular reactor, a batch tank, a planetary mixer, a double arm kneader, a continuous kneader or an extruder. The term “configured to receive a solvent from a container”, as used herein is meant to refer to an arrangement, wherein a reaction vessel or separation vessel is adapted to receive a solvent from a solvent container.

In one embodiment, such solvent container is connected to the reaction vessel/separation vessel by an appropriate pipe, such as a tubing etc.

The methodology according to the present invention results, inter alia, in cellulose, hemicellulose and lignin. Furthermore, the methodology according to the present invention has a stream of phosphate as phosphoric acid as a possible product. The cellulose as well as the hemicellulose can be further processed to result in sugars, for example xylose and glucose. The stream of phosphate is typically recycled to be re-used again in steps a) and b), as outlined above, but can, if desired, also be converted into a fertilizer component. The lignin that results from the methodology according to the present invention can be further used in various applications, outlined further below. Taking xylose as an example of a hemicellulosic-sugar, this is a sugar that was first isolated from wood and hence named for it. Xylose is classified a monosaccharide of the aldo-pentose type, which means that it contains five carbon atoms and includes a formyl functional group. It is the precursive to hemicellulose, one of the main constituents of ligno cellulosic biomass. Like most sugars, it can adopt several structures depending on conditions. With its free carbonyl group, it is a reducing sugar.

The acid catalyzed degradation of hemicellulose provides xylose which can be dehydrated to form furfural, a specialty solvent in industry and a precursor to synthetic polymers.

Xylose is not metabolized by humans. It is absorbed unchanged from the gut, and excreted by the kidneys.

In the following, exemplary uses of xylose according to present invention are outlined:

In animal medicine, xylose is used to test for mal-absorption by administration in water to the patient after fasting. If xylose is detected in blood and/or urine within the next few hours, it has been absorbed by the intestines. Reduction of xylose by catalytic hydrogenation produces the non-cariogenic sugar substitute xylitol.

Xylose—as a sweetener—its use as a sweetener directly is very limited, however, once hydrogenated either chemically or biologically it becomes xylitol which is used as a low glycemic index non-cariogenic sweetener. The actual market for xylitol is actually not very large and can be flooded by xylitol made by chemically hydrogenating xylose from China very quickly.

During the pretreatment of biomass, xylose is or can be dehydrated in the dilute acid process to form furfural. Furfural is a precursor to furan based low cost polymers and resins. Globally, 450,000 to 500,000 tons of furfural are produced each year generally derived from xylose obtained from the hydrolysis of hemicellulose. Furfural is a precursor to polymers, solvents, fragrances, and as an additive to various chemical and polymer formulations.

Xylitol is a sweetener that can be made chemically by hydrogenation of xylose or via fermentation of xylose using a xylitol producing organism. There are a number of naturally occurring organisms that produce xylitol from xylose.

Furthermore, the present invention may result in cellulosic-sugars. Such cellulosic-sugars can be converted into platform molecules through fermentation or chemical transformation. Ethanol is probably the most studied chemical from bio-conversion of sugars. Its uses in biofuel or in the production of ethylene are the largest potential market for this molecule. Di-acids are another type of chemicals that has a high potential for both commodity and specialty chemicals. For instance, succinic acid can be converted in a variety of other chemicals such as 1,4-butanediol or tetrahydrofuran that are building blocks for the production of fibers, polymers or solvents.

Furthermore, the lignin resulting from the present invention may be used as:

(i) dispersants and binders, (ii) emulsifiers, (iii) partially replacement of phenol in phenol-formaldehyde resins and polyurethane foams, (iv) animal health and nutrition, (v) adhesives. (vi) thermoplastic materials, (vii), aromatic monomers (eg benzene, toluene and xylene), (viii) epoxy resins, (ix) precursor to graphitic structures such as activated carbon and carbon fibres and (x) vanillin. An example of another application of lignin is as energy source with a market value determined by its specific heating value.

Furthermore, the methodology according to the present invention also has a stream of phosphate as phosphoric acid as a by-product. As outlined above, the phosphoric acid typically is recycled from the backend of the process to be used in steps a) and b) again, but, alternatively may also be converted to a fertilizer component. If this is desired, the acid can be neutralized with an appropriate base to provide a good fertilizer component. For instance, the acid can be neutralized with potassium hydroxide creating a fertilizer component a phosphorous and potassium for ⅔'s of the (Nitrogen Phosphorus Potassium=NPK) part of the fertilizer. Grass for a lawn might require a 10:10:10 fertilizer or 10 percent Nitrogen, 10 percent Phosphorus, and 10 percent (K) potassium. Alternatively, the acid might be neutralized with an ammonium base providing N and P for the fertilizer component. One might also consider a blend of bases that could include ammonium, potassium, and calcium to actually provide more of a complete fertilizer.

Bases might include NaOH, KOH, NH4OH, Ca(OH)2, Mg(OH)2, and the carbonates for instance of all of the aforementioned materials.

Embodiments according to the present invention relate to an acid and solvent based concept where large quantity of phosphoric acid, ethanol and/or other similar chemical, and water are required at various stages of operation from pretreatment to final products and byproducts. The invention enables the efficient recycling of all the acid and solvents with minimum requirement of makeup chemicals, and also complete recovery of biomass degradation products such as lignin, sugar, etc. from the spent acid and solvent for further use as value added products. The unit operation equipment involved in this recovery process is commercially available and is in operation in the pulp & paper, chemical and mining industries. This invention provides an economical pathway to the commercial scale implementation of the chemical and product biorefinery.

This invention, for the first time, enables the acid and solvent recovery and recycling aspects of the COSLIF based biorefinery. Until now, most of the 2^{nd} generation industries are designed to burn the crude lignin to generate energy in absence of any credible and economic process of isolation and purification for application in value added product development.

Moreover, reference is made to the figures, wherein

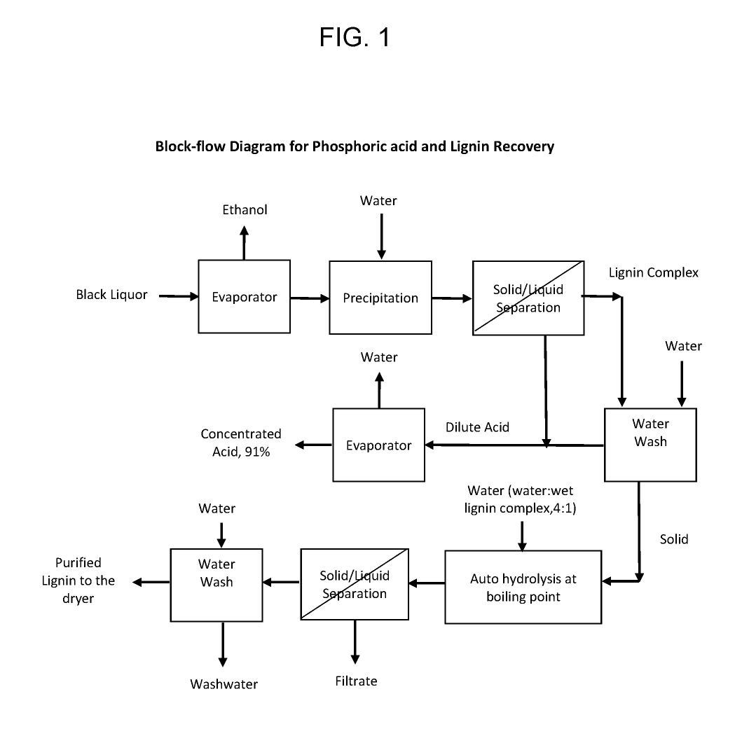

FIG. 1 shows a block flow diagram describing the recovery of phosphoric acid and of lignin according to an embodiment of the present invention, without the use of a flocculant.

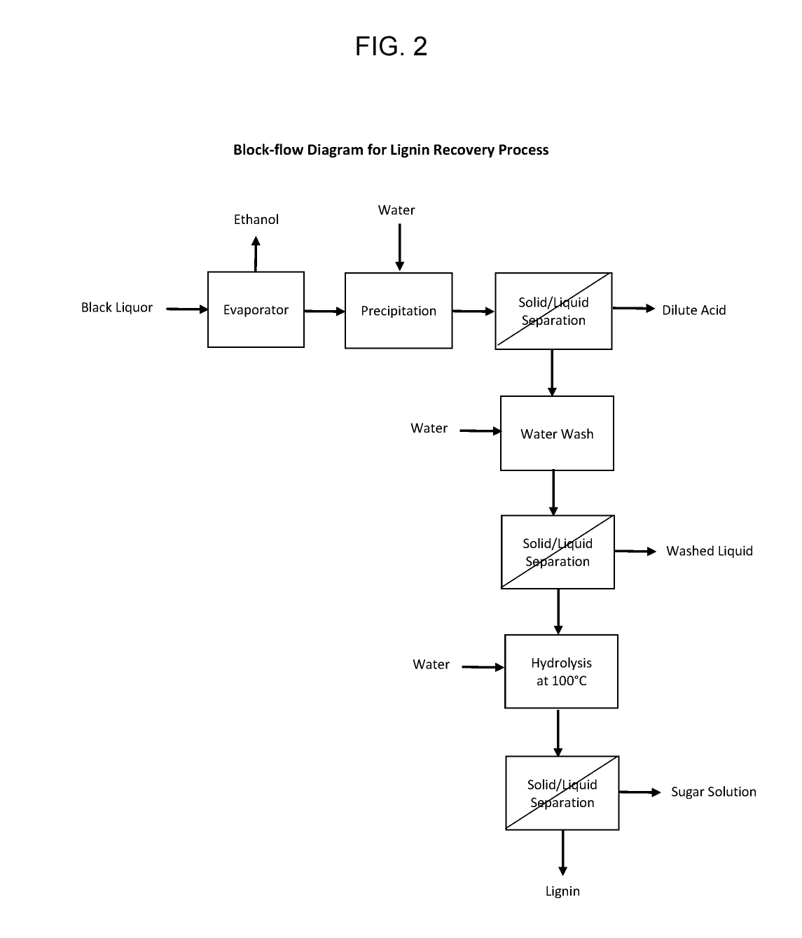

FIG. 2 shows a block flow diagram describing the purification of lignin according to an embodiment of the present invention, without the use of a flocculant,

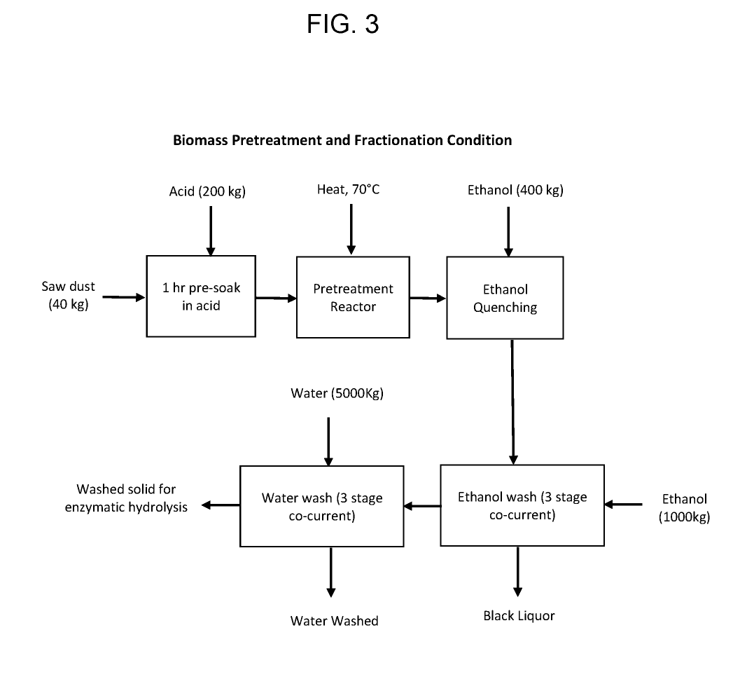

FIG. 3 shows a block flow diagram describing biomass pretreatment and fractionation conditions,

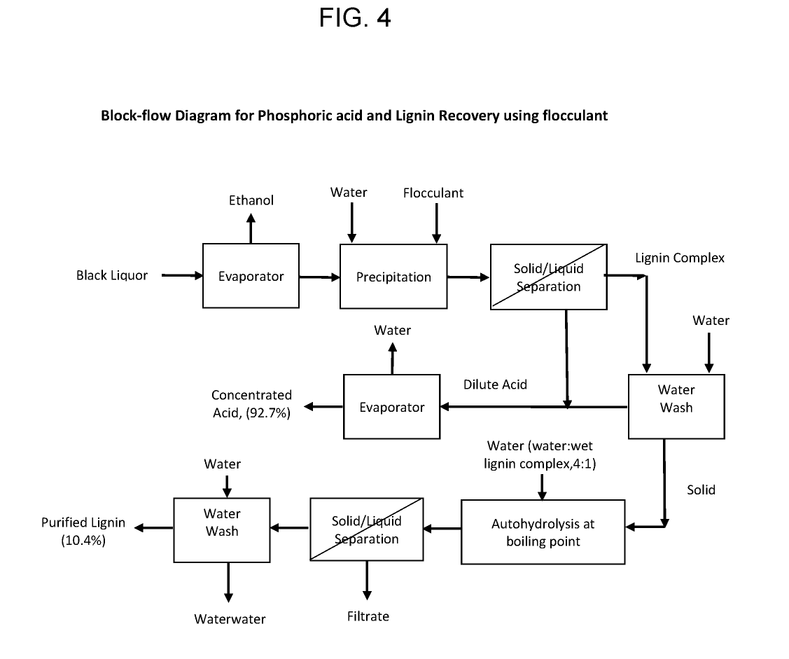

FIG. 4 shows a block flow diagram describing the recovery of phosphoric acid and of lignin according to an embodiment of the present invention involving the use of a flocculant,

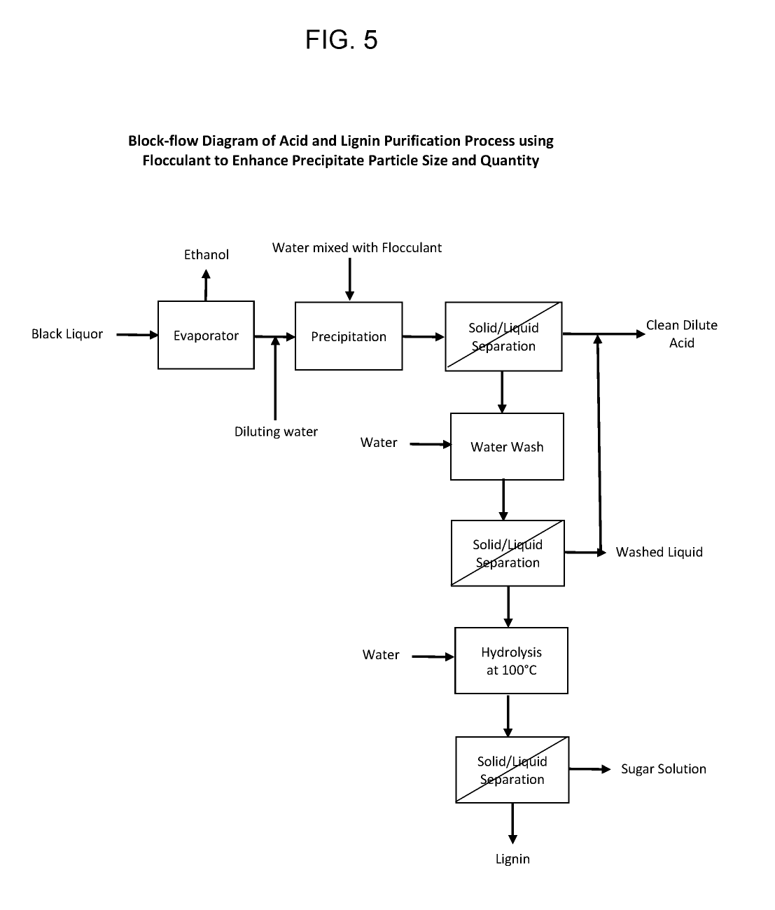

FIG. 5 shows a block flow diagram describing the purification of lignin according to an embodiment of the present invention involving the use of a flocculant,

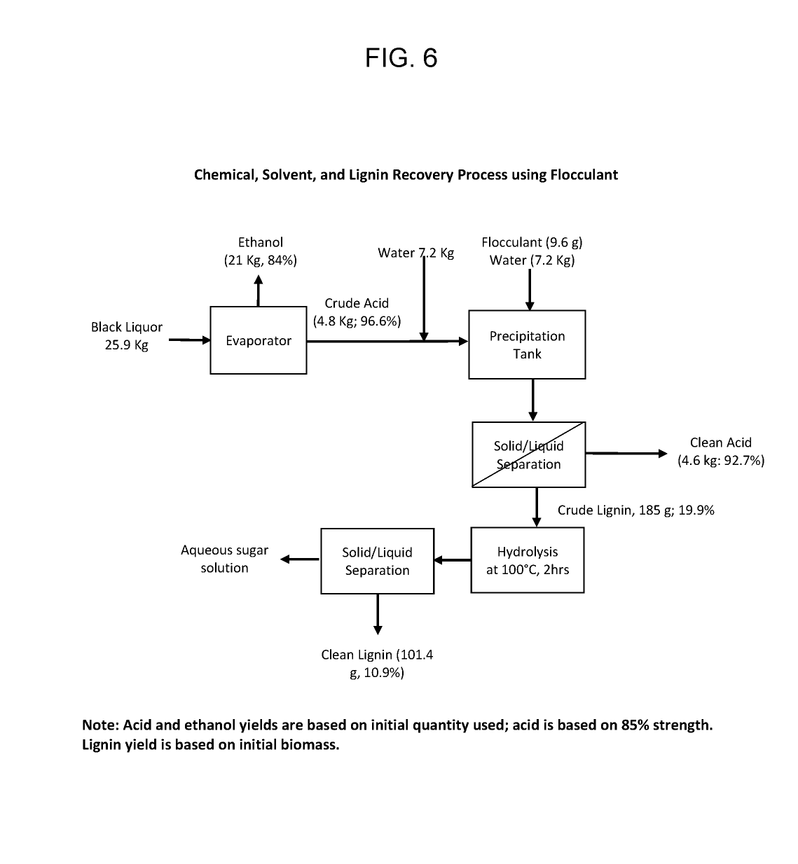

FIG. 6 shows a block flow diagram describing the chemical, solvent and lignin recovery processes in accordance with an embodiment of the present invention, using a flocculant,

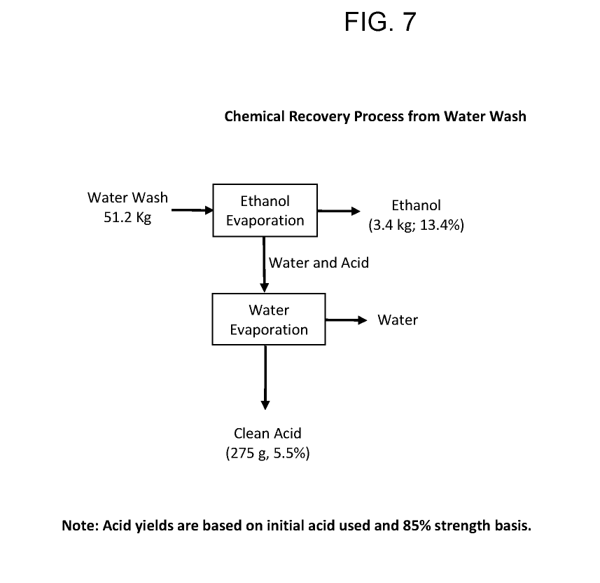

FIG. 7 shows a flow diagram describing the chemical recovery processes from the water wash of the precipitated cellulosic solids in accordance with an embodiment of the present invention.

Furthermore, reference is made to the following exemplary description of embodiments of the present invention which are not intended to limit the present invention.

### A) Embodiments without the Use of Flocculants

An embodiment of the process in accordance with the present invention includes processing of biomass for size reduction, concentrated phosphoric acid treatment of biomass at temperature ranging from 30 to 70° C. for 40 to 80 min, followed by quenching of the reaction and washing of the pretreated solid with ethanol, followed by washing with water before sending the acid and ethanol free solid mass to hydrolysis, preferably enzymatic hydrolysis, and fermentation process. The quenching and ethanol washing steps generate a stream called “black liquor”, whereas the water wash steps generate another stream called water washate.

Black liquor consists of phosphoric acid, ethanol, some water, biomass derived products and extractives. The water washate contains ethanol, water, acid and sugar in monomeric and oligomer form. It is important to recover phosphoric acid, ethanol, and biomass derived products from black liquor to make the whole process economically viable. Most of the used phosphoric acid is in black liquor, significant amount in water washate and very small quantity in washed pretreated biomass.

A process has been developed to separate phosphoric acid from black liquor and purify to a level suitable for reuse in biomass pretreatment process. In an exemplary process, the black liquor was evaporated to collect ethanol as condensate and the residual concentrated black liquor is then mixed with four times of water on a mass basis for precipitation of crude lignin The precipitated particles are very small (about ≦5 μm). The precipitated solid particles are separated by centrifuge and washed with water to remove residual acid along with some water soluble materials. The liquid fraction from solid-liquid separation was concentrated to get an acid stream of 85% strength to be reused in pretreatment process. The precipitated lignin was further purified by hydrolyzing it in acidic medium and by solid/liquid separation and washing. The crude lignin will undergo a mild acidic hydrolysis process to facilitate the conversion of carbohydrate to monomeric sugar. The lignin is maintained as a solid. Following the solid liquid separation, the aqueous sugar solution will go to fermentation process or depending on the sugar concentration, it might be cycled back to water washing with no or minimal additional work. The lignin will be dried for potential use to prepare value added products.

In the phosphoric acid pretreatment process, the black liquor generated through ethanol quenching of the reaction and ethanol washing of residual solid will typically consist of phosphoric acid, ethanol, water and biomass degradation products such as lignin, C5 and C6 monomeric sugars and oligomers, extractives, and other soluble organic and inorganic materials. In an exemplary purification process, the black liquor will then go through an evaporation process where ethanol is removed and passed through a condenser yielding ethanol of 90 to 95% purity. The evaporation process was performed in a rotavapor at temperature in the range of 30 to 75° C. under vacuum at around 15 mbar. The block flow diagram of acid and lignin purification has been shown in FIG. 1.

The viscous concentrated black liquor has a dynamic viscosity of 90 cSt (centistokes) (=90 mPas). This black liquor will then be mixed with water, with water to concentrated black in the ratio of 2:1 to 10:1 as weight percent. This addition of water causes the lignin and minor amounts of carbohydrate to precipitate. Basic compounds were avoided to minimize any salt formation in the acid allowing for more complete recovery.

The particle size of the precipitates is typically in the range of 0.4 to 5 micron. The solid-liquid separation is performed using a centrifuge only since the very small size precipitates passed through and/or blocks the pore to slow the filtration. The filtrate from the solid liquid separation is dilute phosphoric acid, whereas the precipitate is crude lignin along with residual acid, oligomers, carbohydrates, inorganic etc. The precipitates then undergo a water wash to recover some residual acid. The washed acid is then added to the dilute acid stream before going to the evaporator to remove water to produce concentrated acid. The dilute acid was evaporated in rotavapor at around 60 to 90° C. under vacuum at 15 mbar to remove water. The strength of the recovered acid is around 91% well above the fresh acid of 85% strength. This recovered acid after diluting to 85% strength level can be used in the biomass pretreatment step(s) (i.e. steps a) and/or b)). The washed crude lignin is then going to an autohydrolysis process at 100° C. for two hrs. After cooling down, the mass undergoes a solid/liquid separation and washing step. The solid residue is the purified lignin. The filtrate will contain mainly sugar and some soluble materials. The crude lignin compositional analysis shows the presence of ash, the majority of which comes from acid as P₂O₅. As for example, the following compositional analysis presented in Table 1 for crude lignin, washed lignin and hydrolyzed and washed lignin indicates that the precipitated crude lignin and simple washed lignin contains carbohydrate and ash in addition to lignin. The high quantity of ash both in crude lignin and water washed lignin is mostly P₂O₅ coming from residual phosphoric acid adhered to the crude lignin and water washed crude lignin. The hydrolysis of washed crude lignin without addition of any acid, converts most of the carbohydrate into monomer sugar. Through the washing of hydrolyzed material, all sugars and any residual acid go into the aqueous phase leaving behind the purified lignin. The presence of small quantity of ash in purified lignin is the true ash coming from original biomass.

TABLE 2
Compositional analysis of Crude lignin, water washed lignin, hydrolyzed & washed purified lignin

| | Ash | Lignin | Lignin | | | | | | | | |
|---|---|---|---|---|---|---|---|---|---|---|---|
| Types of | (extractive | (AIL) | (ASL) | Glucan | Xylan | Galactan | Arabinan | Manan | Glucuronic | Acetyl | Mass closure |
| lignin | free) (%) | (%) | (%) | (%) | (%) | (%) | (%) | (%) | Acid, (%) | (%) | (%) |
| Crude | 44.6 | 26.3 | 0.1 | 0 | 0 | 0 | 26.2 | 0 | 2.0 | 0.8 | 100 |
| lignin | | | | | | | | | | | |
| Water | 15.6 | 73.8 | 0.2 | 0 | 8.7 | 0 | 0 | 0 | 0.6 | 1.1 | 100 |
| Washed | | | | | | | | | | | |
| Purified | 3.6 | 95.0 | 0.2 | 0 | 0 | 0 | 0 | 0 | 0.0 | 1.2 | 100 |
| Lignin | | | | | | | | | | | |

For simplification, the block-flow diagram for lignin purification and for acid purification is shown in FIG. 2.

The recovered acid was processed at 91% strength, well above 85% strength as required in biomass pretreatment. However, it was not possible to remove all small size, e.g. micron size particles by centrifuge or by other filtration method. As a result, the concentrated recovered acid according to this embodiment still contains a significant quantity of micron size black particles that makes the recovered acid black in color and of higher viscosity than that of fresh phosphoric acid. The viscosity of “fresh”, i.e. pristine, i.e. unused, i.e. freshly manufactured, acid is 47 cSt, whereas for recovered acid it is 56.6 cSt in accordance with this embodiment of the present invention. The need to find some method to transform the small particles into larger particles and to remove all the solid particles to get relatively clean recovered acid with strength greater than 85% and of viscosity similar to pristine acid is addressed further below.

The recycled acid obtained following embodiments of the method according to the present invention has been applied in the pretreatment of biomass and compared with that of fresh acid as well as mixed acid containing 50% recycled acid and 50% fresh acid. The pretreatment condition, washing condition and pretreatment yield has been presented in Table 3.

TABLE 3
Biomass pretreatment condition, washing requirement and biomass yield.

| | | | | | | | Water | |
|---|---|---|---|---|---|---|---|---|
| | | | | | | Ethanol | washing, | |
| | | | | | | washing, | water to | |
| | | Liquid/ | Pretreatment | | Quenching | ethanol:bio- | biomass | |
| | | solid | temperature, | Pretreatment | ethanol:Bio- | mass ratio | ratio, (7 | Pretreatment |
| Biomass | Acid type | ratio | ° C. | time, min | mass | (2 times) | times) | yield, % |
| Sawdust | Recovered | 5:1 | 60 | 60 | 10:1 | 15:1 | 50:1 | 57.5 |
| | Acid (100%) | | | | | | | |
| Sawdust | Fresh acid | 5:1 | 60 | 60 | 10:1 | 15:1 | 50:1 | 58.5 |
| | (100%) | | | | | | | |
| Sawdust | Recovered | 5:1 | 60 | 60 | 10:1 | 15:1 | 50:1 | 60.8 |
| | acid:Fresh | | | | | | | |
| | Acid (50:50) | | | | | | | |

It appears from the pretreatment yield prospective, the recovered acid is quite effective to use in pretreatment process either recovered acid alone or in combination with fresh acid.

The biomass pretreatment and fractionation conditions are shown in FIG. 3.

### B) Embodiments with the Use of Flocculants

As pointed out above, a process has been developed to separate phosphoric acid from black liquor and purify to a level suitable for reuse in biomass pretreatment process. In an exemplary process, the black liquor was evaporated to collect ethanol as condensate and the residual concentrated black liquor is then mixed with four times of water for precipitation of crude lignin. The precipitated particles are very small in size (about ≦5 μm). The precipitated solid particles are separated by centrifuge and washed with water to remove residual acid along with some water soluble materials. The liquid fraction from solid-liquid separation was concentrated to get an acid stream of 85% strength to be reused in pretreatment process. However, the concentrated acid still contains small quantity of small particles which was not possible to remove from dilute acid. This makes the concentrated acid still black and viscosity around 56 cSt (=56 mPas) quite above that of fresh acid.

In order to maximize the quantity of precipitate and enhance the particle size, in one embodiment, the concentrated black liquor is treated with a flocculant, preferably an anionic flocculants more preferably an anionic flocculants solution. Pursuant to such treatment, the quantity of solid fraction and the size of the particle are significantly higher than those obtained by precipitating with water only. The solid/liquid separation can easily be performed by using filter paper of pore size 25 micron under vacuum. The crude lignin was then washed one time with water to remove residual acid. The crude lignin still containing some acid will undergo a mild acidic hydrolysis process to facilitate the conversion of carbohydrate to monomeric sugar. The lignin is maintained as a solid and separated from aqueous solution by filtration.

More specifically, in the phosphoric acid pretreatment process, the black liquor generated through ethanol quenching of the reaction and ethanol washing of residual solid will typically consist of phosphoric acid, ethanol, water and biomass degradation products such as lignin, C5 and C6 monomeric sugars and oligomers, extractives, and other soluble organic and inorganic materials. In an exemplary purification process, the black liquor will go through an evaporation process where ethanol is removed and passed through a condenser yielding ethanol of 90 to 95% purity. The evaporation process was performed in a rotavapor at temperature range 30 to 60° C. under vacuum at around 15 mbar.

The block flow diagram of an exemplary acid and lignin purification process from black liquor using a flocculant has been shown in FIG. 4.

The viscous concentrated black liquor has a dynamic viscosity of about 90 cSt (centistokes) (=90 mPas). In this exemplary purification process, water of about 1 to 3 times at temperature 20 to 30° C. was added to the concentrated black liquor in a container. Another equivalent water quantity was poured in another container. 0.2% of an anionic flocculant (e.g. Nalclear 7763 or Optimer 9825, see Table 1) based on concentrated black liquor were measured and mixed uniformly with water in the 2^{nd} container. All quantities are measured on weight basis. Then the diluted black liquor was poured into the container containing flocculated water and mixed gently to allow formation of flocculated solid particles. The formation of flocculated particle is instantaneous.

The filtrate from the solid liquid separation is dilute phosphoric acid, whereas the precipitate solid is crude lignin along with residual acid, oligomers, carbohydrates, inorganic etc. The precipitate is then undergoing a water wash to recover some residual acid. The washed acid is then added to the dilute acid stream before going to the evaporator to remove water to produce concentrated acid. The dilute acid was evaporated in a rotavapor at around 70 to 90° C. under vacuum at 15 mbar. The strength of the recovered acid is around 91% well above the fresh acid of 85% strength. This recovered acid after diluting to 85% strength level as required by pretreatment condition can be used in the biomass pretreatment step a) or the exposure step b). The recovered acid was processed at 91% strength, well above 85% strength as required in biomass pretreatment. The recovery rate of clean acid is about 94% of the acid used in pretreatment process. The remaining 6% might end up in the filtrate from lignin separation, water wash and small quantity in washed solid residue. The viscosity of recovered acid is around 47.6 cSt (=47.6 mPas) whereas that of fresh acid is 47 cSt (=47 mPas). The color of the recovered acid is lighter than that of recovered acid processed without using flocculants during precipitation of lignin.

The washed crude lignin is then going to an autohydrolysis process at 100° C. for two hrs. After cooling down, the mass undergoes a solid/liquid separation and washing step. The solid residue is the purified lignin. The filtrate will contain mainly sugar and some soluble materials. The crude lignin compositional analysis shows the presence of ash, majority of which coming from acid as P₂O₅. As for example, the following compositional analysis presented in Table 3 for washed crude lignin, and hydrolyzed and washed lignin indicate that the washed lignin contains carbohydrate and ash in addition to lignin. The hydrolysis of washed crude lignin without addition of any acid, should convert most of the carbohydrate into monomer sugar. Through the washing of hydrolyzed material, all sugars and any residual acid go into the aqueous phase leaving behind the purified lignin. However, the compositional analysis of purified lignin shows complete removal of ash but contain significant quantity of sugar, especially xylose. This might be due to incomplete hydrolysis of carbohydrate and/or good washing. This problem can be solved by simply extending the hydrolysis time and improved washing.

TABLE 4
Compositional analysis of water washed crude lignin and hydrolyzed & washed purified lignin.

| | Ash | Lignin | Lignin | | | | | | | | |
|---|---|---|---|---|---|---|---|---|---|---|---|
| Types of | (extractive | (AIL) | (ASL) | Glucan | Xylan | Galactan | Arabinan | Manan | Glucuronic | Acetyl | Mass closure |
| lignin | free) (%) | (%) | (%) | (%) | (%) | (%) | (%) | (%) | Acid, (%) | (%) | (%) |
| Water Washed | 1.1 | 79.3 | 0.2 | 0.0 | 18.0 | 0.0 | 0.5 | 0.0 | 0.6 | 0.3 | 100.0 |
| crude lignin | | | | | | | | | | | |
| processed | 0.0 | 92.4 | 0.2 | 0.0 | 5.9 | 1.2 | 0.3 | 0.0 | 0.0 | 0.0 | 100.0 |
| Lignin | | | | | | | | | | | |

For simplification, the block-flow diagram for lignin purification and for acid purification using a flocculant is shown in FIG. 5.

The biomass pretreatment and fractionation conditions for the embodiments using a flocculant are the same as shown in FIG. 3.

The advantage of using a flocculant over not using a flocculant has been presented in Table 5. In both cases, the starting material is concentrated black liquor after evaporating the ethanol. At 85% strength basis, recovered acid is about 96.6% based on initial acid used in pretreatment. The amount of recovered acid from black liquor is only 87.5% of total acid used in pretreatment and lignin precipitation is performed with water only, whereas the amount of acid is increased to 92.7% when the precipitation is done with water mixed with an anionic flocculant. Without wishing to be bound by any theory, the inventors believe that the recovery of increased amount of lignin and clean acid is due to the formation of large agglomerated crude lignin precipitate with a flocculant suitable for better solid/liquid during filtration in comparison when no flocculant is used. The clean lignin content is 1.5% higher than that of when no flocculant is used.

TABLE 5
Comparison of product recovery from black
liquor with and without a flocculant.

| | Without | With |
|---|---|---|
| Recovered Product | flocculant | flocculant |
| Crude acid, 85% | 96.6% | 96.6% |
| Clean Acid, 85% (based on acid used in | 87.5% | 92.7% |
| pretreatment) | | |
| Processed Lignin (based on biomass) | 9.4% | 10.9% |

The overall recovery of ethanol, phosphoric acid and lignin by accounting the materials both in black liquor and water wash both with and without use of a flocculant in lignin precipitation has been presented in Table 6. The overall acid recovery increased significantly from 93% to 98.2% when using a flocculant. The ethanol recovery remain the same as 97.4% in both cases as a flocculant is not involved in the ethanol recovery process. The purified lignin content increased slightly from 9.4% to 10.9%. The lignin purity is very much related to the extent of hydrolysis of crude lignin at 100° C. It is possible to adjust the lignin purity at any level as required for end use by adjusting the hydrolysis conditions.

TABLE 6
Comparison of product recovery combined from black liquor
and water wash with and without flocculant use.

| | Without | With |
|---|---|---|
| Recovered products | flocculant | flocculant |
| Total acid recovered (based on acid used in | 93% | 98.2% |
| pretreatment) from Black liquor and water wash | | |
| Total ethanol recovered (based on total ethanol | 97.4% | 97.4% |
| used in pretreatment) from Black liquor and | | |
| water wash | | |
| Total processed lignin collected from black | 9.4% | 10.9% |
| liquor (based on biomass) | | |

The block-flow diagrams in FIG. 6 shows the lignin purification process when a flocculant is used in lignin precipitation. The water wash obtained from water washing of pretreated material before sending to enzymatic hydrolysis process, was evaporated with rotavapor to remove ethanol and then water from the acid. The bock flow diagram in FIG. 7 shows the recovery process of acid and ethanol from a water wash of the cellulosic solids.

Furthermore reference is made to the examples which are given to illustrate not to limit the present invention.

### EXAMPLES

### A) Examples without the Use of Flocculant

### Example 1

Baseline pretreatment conditions were used for the preparation of the biomass, quenching of the reaction/precipitation of the cellulose and hemicellulose, ethanol removal and recovery from the acid, lignin precipitation using water, water removal from the dilute acid stream via evaporation, and recovery and reuse of the “used” acid stream.

The conditions of the pretreatment are provided above in Table 3 and the biomass pretreatment and fractionation recipe is given in the following table 7.

TABLE 7
Biomass Pretreatment and Fractionation Recipe.

| | |
|---|---|
| 40 | Kg Sawdust |
| 200 | Kg Acid |
| 1 | Hour presoak in acid |
| 70 | ° C. reaction temperature |
| 400 | Kg ethanol for quenching or precipitation |
| 600 | Kg ethanol for wash step 1 (3 stage co-current) |
| 2000 | Kg water for wash step 2 (3 stage co-current) |

The biomass is mixed with the phosphoric acid (85%) and allowed to sit or soak for 1 to 5 hours. For the acid recovery and recycle work 1 hour presoak was used. Once the presoak period is completed, the acid and biomass is transferred into the reactor (Charles Ross and Sons 200 gallon planetary mixer). The mixture is heated to 70° C. and held at this temperature for 1 hour. At the end of the reaction period the reaction is quenched with 400 Kg of ethanol. Because the acid dissolves the biomass, the quench process actually causes the cellulose and hemicellulose to precipitate as solids (cellulosic solids). The lignin fraction remains soluble in the acid and ethanol solution.

The cellulosic solids are removed from the acid and ethanol solution using filtration. At lab scale, the solids can be readily settled by centrifugation and decantation. At process development scale and pilot scale, a plate and frame filtration system can be used. A decanting or a basket centrifuge could also be used. The solids are washed while in the filter with another 600 Kg of ethanol (ethanol to biomass ratio: 15:1) in a 3 stage co-current wash process to remove all of the acid. The ethanol is removed from the solids using 2000 Kg of water (water to biomass ratio: 50:1) in a 3 stage co-current wash.

The ethanol and phosphoric acid are put together as a single fraction. The water wash stream is retained as a separate fraction.

### Example 2

The ethanol and phosphoric acid stream or “black liquor” is evaporated to remove the ethanol using reduced pressure and temperatures to avoid causing additional reactions with the lignin or to cause it undergo self-condensation reactions. Once the 50° C. ethanol is removed by evaporation, the lignin is precipitated by using an excess (3.5:1 water to concentrated acid solution) of 70° C. water. Once the lignin is precipitated, it is recovered using filtration or centrifugation. The acid can be recovered by evaporation of the water to recover at least 85% phosphoric acid.

The lignin recovered was equivalent to 0.94% of the mass of the input black liquor.

### Example 3

The black liquor stream is evaporated to remove the ethanol using reduced pressure and temperatures to avoid causing additional reactions with the lignin or to cause it undergo self-condensation reactions. Once the 50° C. ethanol is removed by evaporation, the lignin is precipitated by using an excess (3.5:1 water to concentrated acid solution) of 85° C. water. Once the lignin is precipitated, it is recovered using filtration or centrifugation. The acid can be recovered by evaporation of the water to recover at least 85% phosphoric acid. The lignin recovered was equivalent to 0.99% of the mass of the input black liquor.

### Example 4

The black liquor stream is evaporated to remove the ethanol using reduced pressure and temperatures to avoid causing additional reactions with the lignin or to cause it undergo self-condensation reactions. Once the ethanol is removed by evaporation, the lignin is precipitated by an excess (3.5:1 water to concentrated acid solution) of 22° C. water. The acid lignin solution is maintained at 22° C. for the precipitation. Once the lignin is precipitated, it is recovered using filtration or centrifugation. The acid can be recovered by evaporation of the water to recover at least 85% phosphoric acid.

The lignin recovered was equivalent to 2.9% of the mass of the input black liquor.

### Example 5

The black liquor stream is evaporated to remove the ethanol using reduced pressure and temperatures to avoid causing additional reactions with the lignin or to cause it undergo self-condensation reactions. Once the ethanol is removed by evaporation, the lignin is precipitated by using an excess (3.0:1 water to concentrated acid solution) of 22° C. water. The acid lignin solution is maintained at 22° C. for the precipitation. Once the lignin is precipitated, it is recovered using filtration or centrifugation. The acid can be recovered by evaporation of the water to recover at least 85% phosphoric acid.

The lignin recovered was equivalent to 6.3% of the mass of the input black liquor. The conditions for lignin precipitation and the quantity of crude lignin recovered from concentrated black liquor expressed as percentage of lignin present in initial biomass of example 2-5 above are presented in table 8 below:

TABLE 8
Typical conditions for precipitating the lignin
from the phosphoric acid of examples 2-5.

| | | | | Ratio of | | Crude lignin |
|---|---|---|---|---|---|---|
| | Concentrated | Black | | water to | | based on |
| | Black Liquor | liquor | Water | black liquor | | initial lignin |
| | Temperature | mass | Temperature | for lignin | Precipitation | in biomass |
| | (° C.) | (g) | (° C.) | precipitation | Time (min) | (%) |
| Example 2 | 50 | 100 | 70 | 3.5:1 | 5 | 19 |
| Example 3 | 50 | 100 | 85 | 3.5:1 | 5 | 20 |
| Example 4 | 22 | 100 | 22 | 3.5:1 | 5 | 58.6 |
| Example 5 | 22 | 100 | 22 | 3.0:1 | 5 | 126 |

Based on pretreatment conditions, certain amount of lignin in biomass is released in black liquor and the rest still remains with the pretreated biomass. The percentage of crude lignin collected from black liquor is clearly overestimated when compared with the lignin present in original biomass, since the crude lignin contains ash, some biomass derivatives, extractives and other unknown materials and compounds co-precipitated with the lignin from black liquor.

### B) Example Showing the Use of Recycled Acid Cleaned by Using Flocculant for Lignin Precipitation

### Example 6

The recycled acid obtained following embodiments of the method as mentioned in this invention has been applied in pretreatment of biomass (saw dust). The pretreatment condition, washing condition and pretreatment yield has been presented in Table 9. From a pretreatment yield prospective, it appears that recovered acid acts as good as fresh acid. A recovered acid to fresh acid ratio of 93:7 was used (as shown in 1^{st} row of table 9 below) as 93% of acid could be recovered (based on acid used in the pretreatment) from black liquor while 100% fresh acid was used in pretreatment. In a second pretreatment run, only 85% could be recovered (from black liquor) and was tested using the same pretreatment condition adjusting acid composition: 85% recycled and 15% fresh.

TABLE 9
Biomass pretreatment condition, washing requirement and biomass yield.

| | | | | | | | Water | |
|---|---|---|---|---|---|---|---|---|
| | | | | | | Ethanol | washing, | |
| | | | | | | washing, | water to | |
| | | Liquid/ | Pretreatment | | Quenching | ethanol:bio- | biomass | |
| | | solid | temperature, | Pretreatment | ethanol:Bio- | mass ratio | ratio, (7 | Pretreatment |
| Biomass | Acid type | ratio | ° C. | time, min | mass | (2 times) | times) | yield, % |
| Sawdust | Recovered:fresh | 5:1 | 60 | 60 | 6:1 | 6:1 | 6:1 | 66.9 |
| | acid (93:7) | | | | | | | |
| Saw dust | Recovered:Fresh | 5:1 | 60 | 60 | 6:1 | 6:1 | 6:1 | 68.2 |
| | acid (85:15) | | | | | | | |
| sawdust | Fresh acid | 5:1 | 60 | 60 | 6:1 | 6: | 6:1 | 65.0 |
| | (100%) | | | | | | | |

### C) Example with the Use of a Coagulant

### Example 7

Baseline pretreatment conditions were used for the preparation of the biomass, quenching of the reaction/precipitation of the cellulose and hemicellulose, ethanol removal and recovery from the acid, lignin precipitation with water using flocculant, water removal from the dilute acid stream via evaporation, and recovery and reuse of the “used” acid stream. The conditions of the pretreatment are provided above in Table 9 and in Table 10 below.

TABLE 10
Biomass Pretreatment and Fractionation Recipe.

| | |
|---|---|
| 40 | Kg Sawdust |
| 200 | Kg Acid |
| 1 | Hour presoak in acid |
| 70 | ° C. reaction temperature |
| 400 | Kg ethanol for quenching or precipitation |
| 600 | Kg ethanol for wash step 1 (3 stage co-current) |
| 2000 | Kg water for wash step 2 (3 stage co-current) |

The biomass is mixed with the phosphoric acid (85%) and allowed to sit or soak for 1 to 5 hours. For the acid recovery and recycle work 1 hour presoak was used. Once the presoak period is completed, the acid and biomass is transferred into the reactor (Charles Ross and Sons 200 gallon planetary mixer). The mixture is heated to 70° C. and held at this temperature for 1 hour. At the end of the reaction period the reaction is quenched with 400 Kg of ethanol. Because the acid dissolves the biomass, the quench process actually causes the cellulose and hemicellulose to precipitate as solids (cellulosic solids). The lignin fraction remains soluble in the acid and ethanol solution.

The cellulosic solids are removed from the acid and ethanol solution using filtration. At lab scale, the solids can be readily settled by centrifugation and decantation. At process development scale and pilot scale, a plate and frame filtration system can be used. A decanting or a basket centrifuge can also be used. The solids are washed while in the filter with another 600 Kg of ethanol in a 3 stage co-current wash process to remove all of the acid. The ethanol is removed from the solids using 2000 Kg of water in a 3 stage co-current wash.

The ethanol and phosphoric acid are put together as a single fraction. The water wash stream is retained as a separate fraction. The phosphoric acid, ethanol and lignin recovery process has been depicted as a block-flow diagram in FIG. 6. In this process, an anionic flocculant (e.g. Nalclear 7763) was used to facilitate the formation of bigger particle size precipitates that increase the solid/liquid separation efficiency. The lignin processing condition from concentrated biomass (saw dust and corn Stover) using flocculant and lignin yield has been presented in Table 11.

TABLE 11
Lignin processing condition and yield based on lignin in biomass.

| | Conc. | | Conc. black | | | Crude lignin | Clean |
|---|---|---|---|---|---|---|---|
| | black | | liquor to | Washing, | Process | yield, based | lignin |
| Types of | liquor | Flocculant | water ratio | acid to | temp. | on lignin in | yield |
| biomass | (g) | (g) | (mass basis) | water ratio | ° C. | biomass (%) | (%) |
| Saw dust | 100 | 0.2 | 3:1 | 1:1 | 22 | 90.4 | 49.9 |
| Corn Stover | 100 | 0.2 | 3:1 | 1:1 | 22 | 275 | 79.2 |

A typical lignin precipitation condition for recovery and purification of both phosphoric acid and lignin has been shown in Table 11. The typical lignin content of saw dust (mixed hardwood) and corn Stover is 20% and 18% respectively. The amount of crude lignin collected is much higher than the actual lignin in their respective biomass. This implies that some carbohydrate, biomass ash, condensed products and degradation products in the black liquor coming from pretreatment of biomass are co-precipitated with lignin. This is especially true for corn stover due to its high content of ash, hemicellulose, extractives, etc. However, after cleaning of crude lignin through intensive washing and then hydrolyzing the crude lignin to remove all available carbohydrate into sugar form, the percentage of lignin recovered is quite reasonable.

A block-flow diagram in FIG. 6 shows the lignin purification process when a flocculant is used in lignin precipitation.

The water wash obtained from water washing of pretreated material before sending to enzymatic hydrolysis process, was evaporated with rotavapor to remove ethanol and then water from the acid. The acid and ethanol recovery process has been shown as a block-flow diagram in FIG. 7.

The features of the present invention disclosed in the specification, the claims and/or in the accompanying drawings, may, both separately, and in any combination thereof, be material for realizing the invention in various forms thereof.

1. A method for fractionating a lignocellulosic biomass, comprising the steps:
a) providing a lignocellulosic biomass and pretreating said lignocellulosic biomass with phosphoric acid;
b) exposing said pretreated lignocellulosic biomass to phosphoric acid for a defined period of time at a defined temperature range to dissolve cellulosic and/or hemicellulosic components of said biomass;
c) quenching, within the same reaction vessel as for step b), the dissolution of said cellulosic and/or hemicellulosic components, by adding a first solvent to said lignocellulosic biomass, wherein said first solvent is water, an alcohol, an ether, a ketone or a mixture of any of the foregoing, thereby precipitating said cellulosic and hemicellulosic components as solids;
d) separating said precipitated solids of step c) from a liquid fraction comprising said phosphoric acid and said first solvent of step c), washing said separated precipitated solids by a second solvent which is water, an alcohol, an ether, a ketone or a mixture of any of the foregoing, and combining said liquid fraction with said second solvent that has been used for washing said separated precipitated solids, to form a combined liquid fraction, said combined liquid fraction comprising said phosphoric acid, said first and second solvent and lignin components dissolved in any of said phosphoric acid, said first or said second solvent;
e) evaporating said first and said second solvent from said combined liquid fraction, thereby producing a concentrated combined liquid fraction, and, optionally, recycling said first and second solvents, after condensation thereof, to be re-used in step c) and/or d);
f) adding water to said concentrated combined liquid fraction, to precipitate said lignin components dissolved in said concentrated combined liquid fraction, such as lignin, thus producing a precipitate of said lignin components, and a dilute combined liquid fraction, said dilute combined liquid fraction comprising phosphoric acid and water;
g) separating said precipitate of said lignin components from said dilute combined liquid fraction, thus producing a solid crude lignin fraction and, separately therefrom, said dilute combined liquid fraction;
h) concentrating said dilute combined liquid fraction by evaporating said water therefrom, thus producing concentrated phosphoric acid; and
i) recycling said concentrated phosphoric acid of step h) to steps a) and/or b).

2. The method according to claim 1, wherein said evaporation in step e) occurs at a pressure <1 bar and at a temperature below the boiling point(s) of said first and second solvent, preferably <78°, preferably in the range of from 20° C. to 78° C.

3. The method according to claim 1, wherein said water that is added in step f) has a temperature that is equal or higher than the temperature of said concentrated combined liquid fraction and that is in the range of from 20° C. to 90° C.

4. The method according to claim 1, wherein the mass ratio of water to concentrated combined liquid fraction in step f) is in the range of from 1:1 to 10:1, preferably 1:1 to 5:1, preferably 1:1 to 4:1, more preferably 2:1 to 3:1.

5. The method according to claim 1, wherein said concentrated phosphoric acid obtained in step h) is mixed with fresh concentrated phosphoric acid, and is thereafter recycled in step i) as such mixture to be used in step a) and/or b).

6. The method according to claim 1, wherein said first and said second solvent are independently, at each occurrence, selected from water, methanol, ethanol, isopropanol, 1-propanol, 1-butanol, 2-butanol, t-butanol, isobutanol, fatty alcohol, acetone, butanone, pentanone, dimethylether, methylether, diethylether, tetrahydrofurane, and mixtures of any of the foregoing.

7. The method according to claim 1, wherein in step f), additionally, a flocculant is added.

8. The method according to claim 7, wherein said flocculant is an anionic, cationic or non-ionic flocculant, preferably an anionic flocculant or a polyacrylamide-based flocculant, more preferably an anionic polyacrylamide-based flocculant, more preferably Nalclear® or Optimer®, even more preferably Nalclear® 7763 or Optimer® 9825.

9. The method according to claim 7, wherein said flocculant is added in an amount of 0.05 wt. %-2 wt. %, preferably 0.05 wt. %-1 wt. %, more preferably 0.1 wt. % to 0.5 wt. %, with reference to the weight of said concentrated combined liquid fraction.

10. The method according to claim 1, wherein said first and second solvent are the same.

11. The method according to claim 1, wherein, in the pretreatment step a), the weight ratio of said phosphoric acid to said lignocellulosic biomass is in the range of from 1:1 to 13:1, preferably 2:1 to 8:1, more preferably 4:1 to 6:1.

12. The method according to claim 1, wherein said pretreating occurs for a time period of 1 min to 30 h, preferably 1 h to 5 h, and/or at a temperature in the range of from 0° C. to 100° C., preferably 25° C. to 60° C.

13. The method according to claim 1, wherein step b) is performed for a period of 30 min to 5 h, preferably 1 h to 5 h, and at a temperature higher than the temperature of the pretreating step a), preferably in the range of from 40° C. to 90° C., preferably 50° C. to 80° C., more preferably 60° C. to 70° C.

14. The method according to claim 1, wherein the weight ratio of said first solvent to said biomass in step c) is in the range of from 12:1 to 5:1, preferably 10:1 to 6:1.

15. The method according to claim 1, wherein the weight ratio of said second solvent to said biomass in step d) is 30:1 to 2:1, preferably 25:1 to 6:1(.

16. The method according to claim 1, comprising the additional first step: washing the precipitated solids washed in step d), by water at a weight ratio of water:biomass of 200:1 to 50:1, preferably 150:1 to 100:1.

17. The method according to claim 16, comprising the additional second step: subjecting the water washed solids of the additional first step to hydrolysis, preferably enzymatic hydrolysis, more preferably by cellulase(s) and/or hemicellulase(s), and subjecting the product of such hydrolysis subsequently to fermentation.

18. The method according to claim 1, comprising the additional step: subjecting the solid crude lignin fraction of step g) to an acidic hydrolysis using water, preferably at a temperature in the range of from 50° C. to 100° C., said acidic hydrolysis being optionally preceded by one or several washing steps using water, said hydrolysis resulting in a solid fraction and a liquid fraction, separating said solid fraction and said liquid fraction from each other, washing the solid fraction and drying it, wherein said liquid fraction is an aqueous solution of sugars, including sugar monomers and oligomers, and said solid fraction is purified lignin.
